SEQUENCE LISTING (13 sequences)

SEQ ID NO: 1 - RNA, 18 nt, Homo sapiens
gucccuguucaggcgcca

SEQ ID NO: 2 - RNA, 22 nt, Homo sapiens
uguaaacauccucgacuggaag

SEQ ID NO: 3 - RNA, 22 nt, Homo sapiens
aguuuugcauaguugcacuaca

SEQ ID NO: 4 - RNA, 23 nt, Homo sapiens
aaggagcuuacaaucuagcuggg

SEQ ID NO: 5 - RNA, 24 nt, Homo sapiens
uuaaugcuaaucgugauagggguu

SEQ ID NO: 6 - RNA, 23 nt, Homo sapiens
aguuuugcagguuugcauccagc

SEQ ID NO: 7 - RNA, 22 nt, Homo sapiens
cugugcgugugacagcggcuga

SEQ ID NO: 8 - RNA, 22 nt, Homo sapiens
acuggacuuggagucagaaggc

SEQ ID NO: 9 - RNA, 23 nt, Homo sapiens
ugugcuugcucgucccgcccgca

SEQ ID NO: 10 - RNA, 22 nt, Homo sapiens
ucguaccgugaguaauaaugcg

SEQ ID NO: 11 - RNA, 20 nt, Homo sapiens
agagguauagggcaugggaa

SEQ ID NO: 12 - RNA, 22 nt, Homo sapiens
cugugcgugugacagcggcuga

SEQ ID NO: 13 - RNA, 20 nt, Homo sapiens
gugcauugcuguugcauugc

(19) United States
(12) Patent Application Publication
(10) Pub. No.: US 2020/0248268 A1
(43) Pub. Date: Aug. 6, 2020

(54) Method For Estimating A Risk For A Subject Suffering From Urothelial Carcinoma And Kit Thereof

(71) Applicants: National Central University, Taoyuan City (TW); China Medical University, Taichung City (TW)

(72) Inventors: Nian-Han Ma, Taoyuan City (TW); Chiu-Chin Huang, Taichung City (TW); Chao-Hsiang Chang, Taichung City (TW); Chien-Lung Chen, Taoyuan City (TW); An-Lun Li, Taoyuan City (TW)

(21) Appl. No.: 16/658,754

(22) Filed: Oct. 21, 2019

(30) Foreign Application Priority Data
Jan. 31, 2019 (TW) 108103917

(51) Int. Cl. C12Q 1/6886

(52) U.S. Cl. CPC C12Q 1/6886; C12Q 2600/112; C12Q 2600/178

(57) ABSTRACT

A method for estimating a risk for a subject suffering from an urothelial carcinoma is provided, including measuring an expression level of at least one miRNA in a sample of the subject, and the miRNA is selected from miR-1274, miR-19a, miR-30, and miR-708; and comparing the expression level of the same miRNA in the sample to that of a control for estimating whether the subject has the risk of suffering from the urothelial carcinoma or not. A kit for estimating a risk for a subject suffering from an urothelial carcinoma is also provided, including at least one reagent for detecting an expression level of at least one miRNA as above mentioned in a sample of the subject.

### CROSS-REFERENCE TO RELATED APPLICATION

This application claims priority to Taiwan Application Serial Number 108103917, filed on Jan. 31, 2019, which is herein incorporated by reference in its entirety.

### BACKGROUND

### Field of Invention

The present invention relates to an estimating method and kit thereof. More particularly, the present invention relates to a method for estimating a risk for a subject suffering from urothelial carcinoma and kit thereof.

### Description of Related Art

Urothelial carcinoma (UC) is a particular urological cancer type of bladder cancer in Taiwan, the incidence rate of UC increases from 55% to 71% as patients with chronic kidney disease (CKD) deteriorate from stage 3 to stage 5. This phenomenon indicates that the occurrence of urothelial carcinoma has a positive relationship with the stages of the chronic kidney disease. As of 2015, the global prevalence rate of chronic kidney disease is 8% to 16%. In addition to medical history inquiry and physical examination, urine test, urine cytology and urography are also important for diagnosing the urothelial carcinoma. The initial symptoms of urothelial carcinoma are not obvious, such as hematuria and low back pain, so that the cancer is often more advanced at the time of confirmed diagnosis. Since there is no suitable tumor marker for diagnosis and follow-up treatment, the disease is often found late and mortality is high.

Therefore, developing biomarkers for detecting urothelial carcinoma is needed, and the disadvantage of the prior art should be resolved.

### SUMMARY

The present disclosure provides a method and a kit for estimating a risk for a subject suffering from an urothelial carcinoma, a highly accurate detection effect is achieved in a non-invasive way.

The present disclosure provides the method for estimating a risk for a subject suffering from an urothelial carcinoma, comprising: measuring an expression level of at least one micro ribosomal nucleic acid (miRNA) in a sample of the subject, wherein the miRNA is selected from the group consisting of miR-1274, miR-19a, miR-30, and miR-708; and comparing the expression level of the miRNA in the sample to that of a same miRNA of a control, wherein a decrease in the expression level of the miRNA selected from the group consisting of miR-1274, miR-30 and miR-708 and/or an increase in the expression level of miR-19a in the sample from the subject, relative to that of the control, estimates the subject being of a risk of suffering from the urothelial carcinoma.

In one embodiment, the control is obtained from a group of subjects that does not have the urothelial carcinoma.

In one embodiment, the subject is a patient receiving peritoneal dialysis.

In one embodiment, the sample comprises ascites, blood, urine, feces, gastric juice, bile, or a combination thereof.

The present disclosure also provides a method for estimating a risk of an urothelial carcinoma for a subject with chronic kidney disease, the method comprising: measuring an expression level of at least one miRNA in a sample of the subject with chronic kidney disease, wherein the miRNA is selected from the group consisting of miR-1274, miR-19a, miR-30, miR-708, miR-155, miR-19b, miR-210, miR-378, and miR-636; and comparing the expression level of the miRNA in the sample to that of a same miRNA of a control, wherein a decrease in the expression level of the miRNA selected from the group consisting of miR-1274, miR-30, miR-155, miR-19b, miR-210, miR-378, and miR-636 and/or an increase in the expression level of the miRNA selected from the group consisting of miR-19a and miR-708 in the sample from the subject with chronic kidney disease, relative to that of the control, estimated the subject being of a risk of suffering from the urothelial carcinoma.

In one embodiment, the control is obtained from a group of subjects with chronic kidney disease that does not have the urothelial carcinoma.

In one embodiment, the subject with chronic kidney disease is a patient receiving peritoneal dialysis.

In one embodiment, the sample comprises ascites, blood, urine, feces, gastric juice, bile, or a combination thereof.

The present disclosure also provides a method for estimating a risk for a subject suffering from an urothelial carcinoma, the method comprising: measuring expression levels of a plurality of miRNAs in a sample of the subject, wherein the plurality of miRNAs comprise miR-1274, miR-19a, miR-30, and miR-708; calculating the expression levels of the plurality of miRNAs to obtain a prediction score S; providing a reference value S0; and estimating the risk for the subject suffering from the urothelial carcinoma, wherein when the prediction score S is more than or equal to the reference value S0, the subject is estimated having the risk of suffering from the urothelial carcinoma.

In one embodiment, the prediction score S is obtained by calculating the expression levels of the plurality of miRNAs with an equation below: prediction score S=−2.061+(1.698*A)+(1.300*B)+(0.861*C)+(1.330*D) equation (1), wherein when the expression level of miR-1274 is less than or equal to 34.61, A=1, when the expression level of miR-1274 is more than 34.61, A=0; when the expression level of miR-19a is more than or equal to 0.0002243, B=1, when the expression level of miR-19a is less than 0.0002243, B=0; when the expression level of miR-30 expression is less than or equal to 3.798, C=1, when the expression level of miR-30 is more than 3.798, C=0; and when the expression level of miR-708 is more than or equal to 2.235*10⁻⁷, D=1, when the expression level of miR-708 is less than 2.235*10⁻⁷, D=0.

In one embodiment, the step of providing the reference value S0 comprises: measuring expression levels of a plurality of miRNAs in samples of a group of subjects with and without the urothelial carcinoma, wherein the plurality of miRNAs comprise miR-1274, miR-19a, miR-30, and miR-708; and calculating the expression levels of the plurality of miRNAs in the samples of the group of subjects with and without the urothelial carcinoma by a receiver operating characteristic curve to obtain a cutoff value as the reference value S0.

In one embodiment, the plurality of miRNAs further comprises miR-155, miR-19b, miR-210, miR-378, and miR-636.

In one embodiment, the prediction score S is obtained by calculating the expression levels of the plurality of miRNAs with an equation below: prediction score S=−3.471+(1.258*A)+(0.590*B)+(0.327*C)+(1.042*D)−(0.561*E)+(1.605*F)−(0.172*G)+(0.413*H)+(1.5411) equation (2), wherein when the expression level of miR-1274 is less than or equal to 34.61, A=1, when the expression level of miR-1274 is more than 34.61, A=0; when the expression level of miR-19a is more than or equal to 0.0002243, B=1, when the expression level of miR-19a is less than 0.0002243, B=0; when the expression level of miR-30 is less than or equal to 3.798, C=1, when the expression level of miR-30 is more than 3.798, C=0; when the expression level of miR-708 is more than or equal to 2.235*10⁻⁷, D=1, when the expression level of miR-708 is less than 2.235*10⁻⁷, D=0; when the expression level of miR-155 is less than or equal to 1.227, E=1, when the expression level of miR-155 is more than 1.227, E=0; when the expression level of miR-19b is less than or equal to 0.3685, F1, when the expression level of miR-19b is more than 0.3685, F=0; when the expression level of miR-210 is less than or equal to 1.797, G=1, when the expression level of miR-210 is more than 1.797, G=0; when the expression level of miR-378 is less than or equal to 0.7642, H=1, when the expression level of miR-378 is more than 0.7642, H=0; and when the expression level of miR-636 is less than or equal to 0.5524, I=1, when the expression level of miR-636 is more than 0.5524, I=0.

In one embodiment, the step of providing the reference value S0 comprises: measuring expression levels of a plurality of miRNAs in samples of a group of subjects with and without the urothelial carcinoma, wherein the plurality of miRNAs comprise miR-1274, miR-19a, miR-30, miR-708, miR-155, miR-19b, miR-210, miR-378, and miR-636; and calculating the expression levels of the plurality of miRNAs in the samples of the group of subjects with and without the urothelial carcinoma by a receiver operating characteristic curve to obtain a cutoff value as the reference value S0.

In one embodiment, the subject is a patient receiving peritoneal dialysis or with chronic kidney disease.

In one embodiment, the sample comprises ascites, blood, urine, feces, gastric juice, bile, or a combination thereof.

The present disclosure also provides a kit for estimating a risk for a subject suffering from an urothelial carcinoma, the kit comprising: at least one reagent, the reagent using for detecting an expression level of at least one miRNA in a sample of the subject, wherein the miRNA is selected from the group consisting of miR-1274, miR-19a, miR-30, miR-708, miR-155, miR-19b, miR-210, miR-378, and miR-636.

In one embodiment, the kit comprises a plurality of reagents using for detecting expression levels of the miRNAs in the sample of the subject, wherein the miRNAs are miR-1274, miR-19a, miR-30, and miR-708.

In one embodiment, the kit comprises a plurality of reagents using for detecting expression levels of the miRNAs in the sample of the subject, wherein the miRNAs are miR-1274, miR-19a, miR-30, miR-708, miR-155, miR-19b, miR-210, miR-378, and miR-636.

In one embodiment, the reagent comprises a pair of primers, a probe, or a combination thereof.

### BRIEF DESCRIPTION OF THE DRAWINGS

The invention can be more fully understood by reading the following detailed description of the embodiment, with reference made to the accompanying drawings as follows.

FIG. 1 shows a flow chart illustrating a screening process for detecting miRNAs in an urothelial carcinoma according to one embodiment of the present disclosure.

FIG. 2 shows expression levels of miRNA-1274a in the sample of control group, chronic kidney disease (CKD) group, and urothelial carcinoma (UC) group according to one embodiment of the present disclosure.

FIG. 3 shows expression levels of miRNA-19a-5p in the sample of control group, CKD group, and UC group according to one embodiment of the present disclosure.

FIG. 4 shows expression levels of miRNA-30a-5p in the sample of control group, CKD group, and UC group according to one embodiment of the present disclosure.

FIG. 5 shows expression levels of miRNA-708-5p in the sample of control group, CKD group, and UC group according to one embodiment of the present disclosure.

FIG. 6 shows expression levels of miRNA-155-5p in the sample of control group, CKD group, and UC group according to one embodiment of the present disclosure.

FIG. 7 shows expression levels of miRNA-19b-1-5p in the sample of control group, CKD group, and UC group according to one embodiment of the present disclosure.

FIG. 8 shows expression levels of miRNA-210-3p in the sample of control group, CKD group, and UC group according to one embodiment of the present disclosure.

FIG. 9 shows expression levels of miRNA-378a-3p in the sample of control group, CKD group, and UC group according to one embodiment of the present disclosure.

FIG. 10 shows expression levels of miRNA-636 in the sample of control group, CKD group, and UC group according to one embodiment of the present disclosure.

FIG. 11 shows the result of expression levels of four miRNAs analyzing by the ROC curve at the same time.

FIG. 12 shows the results of expression levels of nine miRNAs analyzing by the ROC curve at the same time.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

The present disclosure provides a method for estimating a risk for a subject suffering from urothelial carcinoma. In one embodiment, suitable situations for estimating the risk of urothelial carcinoma in the subject by the method of the present disclosure may include urothelial carcinoma induced by long-term peritoneal dialysis or chronic kidney disease, or having urothelial carcinoma without any disease.

The subject may include, but not limited to, human, orangutan, monkey, cat, dog, rabbit, guinea pig, rat or mouse. In one embodiment, the subject may be a patient with dialysis.

Chronic kidney disease is closely related to urothelial carcinoma, and the definition and staging criteria for chronic kidney disease were established in 2002 by the Kidney Disease Outcome Quality Initiative (KDOQI). Chronic kidney disease refers to renal damage or glomerular filtration rate (GFR)<60 mL/min/1.73 m² for more than three months, and chronic kidney disease is divided into five stages according to GFR, as shown in Table 1.

TABLE 1
The stages of chronic kidney disease

| | GFR | |
|---|---|---|
| Stages | (mL/min/1.73 m²) | Description |
| I | ≥90 | Normal or higher than normal |
| II | 60-89 | Mild loss |
| IIIa | 45-59 | Mild to moderate loss |
| IIIb | 30-44 | Moderate to severe loss |
| IV | 15-29 | Severe loss |
| V | <15 | renal failure |

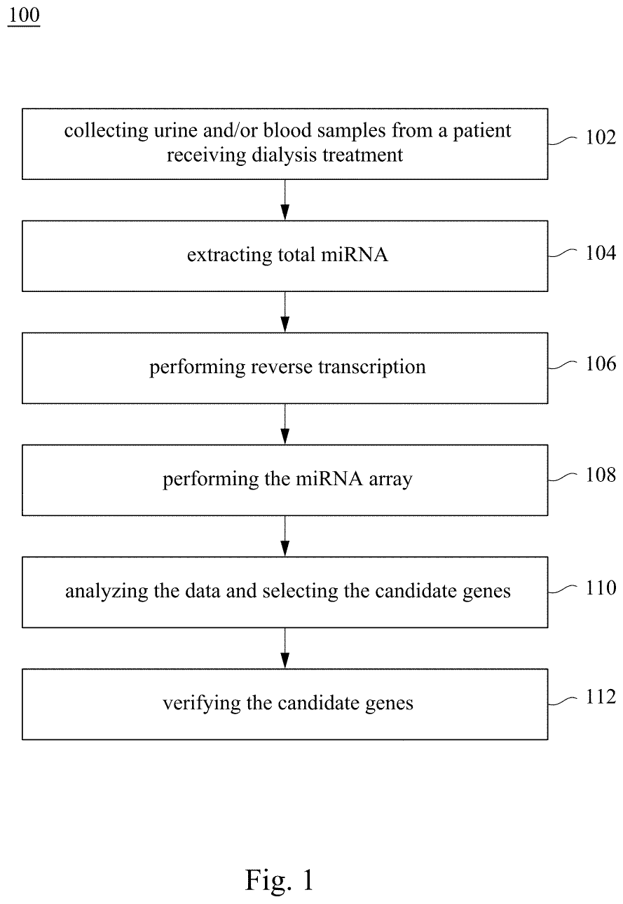

FIG. 1 is a flow chart for screening miRNAs associated with urothelial carcinoma.

The method 100 begins at step 102 collecting urine and/or blood samples from a patient receiving dialysis treatment. Because this group of patients had a high incidence of urothelial carcinoma, the differences of the expression levels of miRNAs between the patients with no urothelial carcinoma (control group and/or CKD group) and the patients with urothelial carcinoma (UC group) were compared.

In some embodiments, the control group obtained a sample from a subject who did not ever have the urothelial carcinoma. In one embodiment, the control group is a normal subject.

Then, step 104 is to extract total miRNA from the urine and/or blood sample. MiRNA is stable in human tissues and cell samples, and miRNA is not easily degraded. MiRNA can be detected in many body fluids such as blood, saliva, urine, and ascites. MiRNA in the patient's urine and/or blood samples was detected in some examples of the present disclosure.

In some embodiments, the “micro ribosomal nucleic acid (abbreviated microRNA or miRNA)” refers to a small non-coding RNA molecule containing about 19 to 25 nucleotides. MiRNA can recognize messenger RNA (mRNA) because of sequences specificity, so that the expression of the gene is regulated by inhibiting the transcription of the mRNA or degrading the mRNA.

After that, step 106 is to reverse transcribe the total miRNA into complementary DNA (cDNA).

Next, step 108 is to perform the miRNA array. The reverse transcription cDNA was detected by the miRNA array chip, and the chip can detect the expression of miRNAs. For example, the chip of TaqMan® Array Human MicroRNA A Cards v2.0 can be used in detecting nearly 380 kinds of human miRNAs expression, and the cycle thresholds (CT) of the cDNAs of the target miRNAs can be obtained.

Further, step 110 is to analyze the data and select the candidate genes. According to the result of the miRNA array, certain miRNAs having significant different expression levels of the between patients with and without urothelial carcinoma can be selected. For example, some certain miRNAs were highly expressed in samples of patients with urothelial carcinoma, and some certain miRNAs were less expressed in samples of patients with urothelial carcinoma. These certain miRNAs can be used as candidate genes for the detection of urothelial carcinoma.

Furthermore, step 112 is to verify the candidate genes. For example, the expression levels of the candidate genes between the patients with and without urothelial carcinoma were measured by real-time polymerase chain reaction (real-time PCR).

In some embodiments, the method of measuring the expression level of miRNA can be performed by, for example, quantitative or semi-quantitative real-time PCR, northern blotting analysis, or liquid hybridization.

In some embodiments, calculating the normalized CT value includes two methods. One was normalized by U6 small nuclear RNA (RNU6). This method used the CT value of RNU6 in the miRNA array as a reference, and the expression level of miRNA was transferred by the formula 2^{-ΔCT}, wherein the ΔCT was obtained from “subtracting the CT value of RNU6 from the CT value of the cDNA of the target miRNA.” The other was that the average CT value of all detected miRNAs in the miRNA array was being as a reference. The expression level of miRNA was transferred by the formula 2^{-ΔCT}, wherein the ΔCT was obtained from “subtracting the average CT value from the CT value of the cDNA of the target miRNA.”

In the present disclosure, the sensitivity and specificity of urothelial carcinoma detection can be obtained from the ROC curve. For example, the ROC curve was drawn by the software “Prism” through inputting the normalized data of the expression level of the miRNA, and the default value was used in the calculation portion. Then, the relative value of the maxima likelihood ratio was chosen as a cutoff value, and the sensitivity and specificity were obtained by the cutoff value.

In addition, some examples further include testing different combinations of miRNAs. As for calculating the expression level of each miRNA, the model equation was established by the multivariate logistic regression to estimate the risk of urothelial carcinoma.

In some embodiment, the present disclosure provides a use of miRNA detecting reagent for manufacture of a kit for risk determination of urothelial carcinoma, wherein the miRNA is selected from group consisting of miR-1274, miR-19a, miR-30, miR-708, miR-155, miR-19b, miR-210, miR-378, miR-636, and combinations thereof.

In some embodiment, the detected sequences of miRNAs are derived from human, wherein miR-1274 is hsa-miR-1274a (SEQ ID NO. 1), miR-30 is hsa-miR-30a-5p (SEQ ID NO. 2), miR-19a is hsa-miR-19a-5p (SEQ ID NO. 3), miR-708 is hsa-miR-708-5p (SEQ ID NO. 4), miR-155 is hsa-miR-155-5p (SEQ ID NO. 5), miR-19b is hsa-miR-19b-1-5p (SEQ ID NO. 6), miR-210 is hsa-miR-210-3p (SEQ ID NO. 7), miR-378 is hsa-miR-378a-3p (SEQ ID NO. 8), and miR-636 is hsa-miR-636 (SEQ ID NO. 9). The miRNAs and its miRBase Mature sequence accession number are hsa-miR-30a-5p (MIMAT0000087), hsa-miR-19a-5p (MIMAT0004490), hsa-miR-708-5p (MIMAT0004926), hsa-miR-155-5p (MIMAT0000646), hsa-miR-19b-1-5p (MIMAT0004491), hsa-miR-201-3p (MIMAT0000267), hsa-miR-378a-3p (MIMAT0000732), and hsa-miR-636 (MIMAT0003306).

In some embodiment, the kit may be in the form of a reagent kit. The kit further includes common reagents for PCR, such as primers or probes for detecting miRNA, buffer, deoxy-ribonucleotide triphosphate (dNTP), magnesium chloride, distilled water, and Taq polymerase.

In some embodiment, the probes or primers may be fixed on the solid support, such as chip.

### EXAMPLE 1

The prediction of urothelial carcinoma by single miRNA

1.1 Collecting Specimens

Specimens of 50 normal subjects, specimens of 111 CKD patients, and specimens of 111 CKD patients with urothelial carcinoma (Table 2) were collected for further analyzing the correlation between miRNAs and urothelial carcinoma. The expression level of the miRNA was analyzed between UC group (the patients already diagnosed with chronic kidney disease and urothelial carcinoma) and CKD group (CKD without suffering from urothelial carcinoma), or UC group and control group (normal subjects), and the miRNAs used as a biomarker for screening urothelial carcinoma was estimated.

TABLE 2
The specimen information in each group

| | Control group | | | CKD group | | | UC group | | | P |
|---|---|---|---|---|---|---|---|---|---|---|
| | Number | AVG | SD | Number | AVG | SD | Number | AVG | SD | value |
| Age | 50 | 62 | 14 | 111 | 64 | 11 | 111 | 66 | 11 | 0.051^{a} |
| Female | 18 | | | 37 | | | 33 | | | 0.707^{a} |
| Male | 32 | | | 74 | | | 78 | | | |

CKD stages

| | | | |
|---|---|---|---|
| I | 20 | 19 | 0.882^{b} |
| II | 21 | 22 | |
| III | 29 | 35 | |
| IV | 22 | 20 | |
| V | 19 | 15 | |

AVG: average;
SD: standard deviation;
^{a}One-way ANOVA test;
^{b}Pearson Chi-Square test.

1.2 Extraction and Quantification of miRNA

In a conventional method, total miRNA in the patient's urine or blood was extracted, and 600 ng of the total miRNA was reverse-transcribed following by the protocol of TaqMan® MicroRNA Reverse Transcriptase to obtain cDNAs. The cDNAs were then preceded with miRNA array according to the protocol provided by the TaqMan® Array Human MicroRNA A Cards v2.0. Specifically, the fluorescent probes provided by the TaqMan® were used to detect the cDNAs of: miR-1274 (e.g. SEQ ID NO. 1), miR-30 (e.g. SEQ ID NO. 2), miR-19a (e.g. SEQ ID NO. 3), miR-708 (e.g. SEQ ID NO. 4), miR-155 (e.g. SEQ ID NO. 5), miR-19b (e.g. SEQ ID NO. 6), miR-210 (e.g. SEQ ID NO. 7), miR-378 (e.g. SEQ ID NO. 8), miR-636 (e.g. SEQ ID NO. 9), miR-126-3p (e.g. SEQ ID NO. 10; MIMAT0000445), miR-202-3p (e.g. SEQ ID NO. 11; MIMAT0002811), miR-210-3p (e.g. SEQ ID NO.12; MIMAT0000267), and miR-33b-5p (e.g. SEQ ID NO. 13; MIMAT0003301), and the fluorescent substances were released while an amplification reaction was performed. The fluorescence intensities were detected to obtain the CT values of cDNAs of miR-1274, miR-19a, miR-30, miR-708, miR-155, miR-19b, miR-210, miR-378, and miR-636. Finally, RNU6 was used for normalization, ACT was obtained from subtracting the CT value of RNU6 from the CT value of the cDNA of the miRNA, and the expression level was converted from the logarithmic value of formula 2^{-ΔCT}.

Estimating a Risk for a Subject Suffering from Urothelial Carcinoma

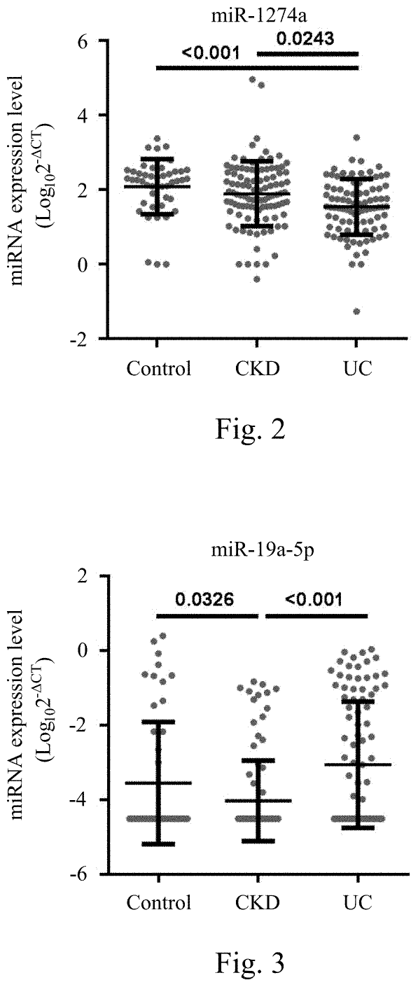

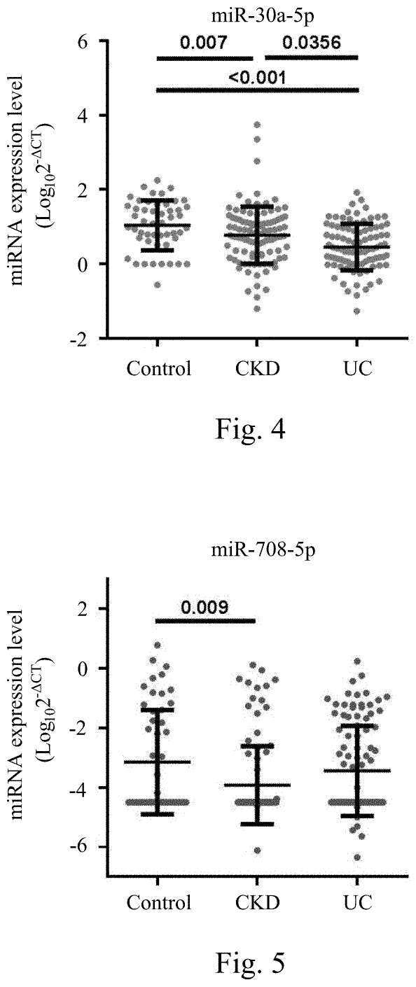

FIGS. 2 to 5 were scatter plots that respectively showed four miRNAs miR-1274, miR-19a, miR-30 and miR-708 extracted from urine in the control, CKD and UC groups. The numbers showed on the top of each figure indicated the statistical P values between the control group and the CKD group, the control group and the UC group, or the CKD group and the UC group. When P<0.05, P<0.01 or P<0.001, the P values were considered having a significant difference. As shown in FIGS. 2, 4, and 5, the expression levels of miR-1274, miR-30, and miR-708 in UC group were less than the control group. However, FIG. 3 showed that the expression level of miR-19a in the UC group was more than that in the control group. Thus, at least one of the miRNAs miR-1274, miR-19a, miR-30, or miR-708 can be used as a biomarker to estimate the risk for the subject suffering from urothelial carcinoma.

Estimating the Risk of Urothelial Carcinoma in Patients with Chronic Kidney Disease

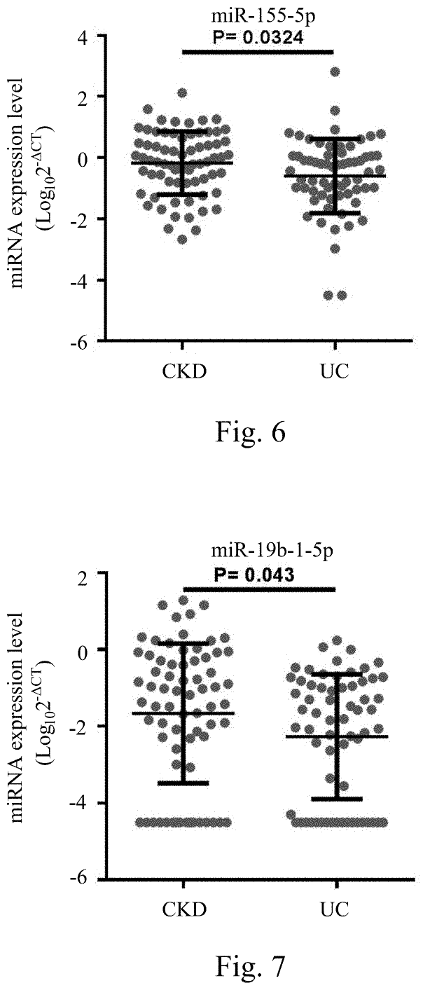

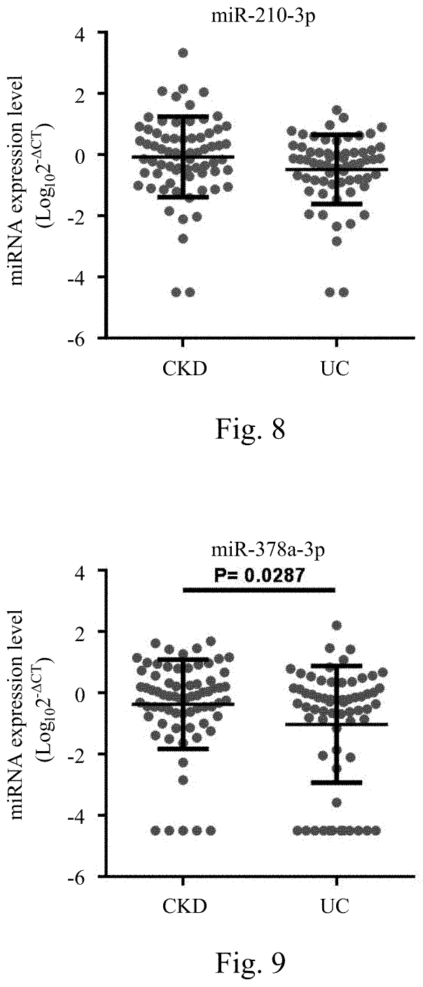

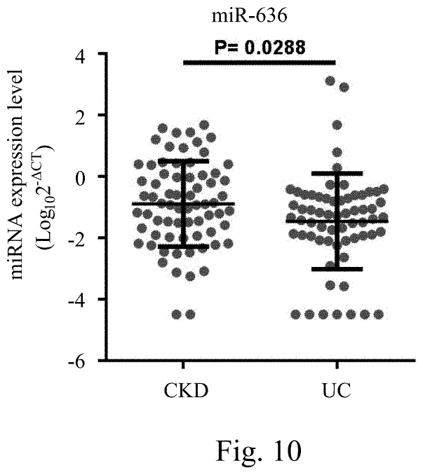

As shown in FIGS. 2 to 10, wherein FIGS. 6 to 10 were scatter plots respectively show five miRNAs miR-155, miR-19b, miR-210, miR-378, and miR-636 extracted from blood in the control, CKD, and UC groups. As shown in FIGS. 2, 4 and 6 to 10, the expression levels of miR-1274, miR-30, miR-155, miR-19b, miR-210, miR-378, and miR-636 in UC group were less than the control group. However, FIGS. 3 and 5 showed that the expression levels of miR-19a and miR-708 in the UC group were more than that in the control group. Thus, at least one of the miRNAs miR-1274, miR-19a, miR-30, miR-708, miR-155, miR-19b, miR-210, miR-378, or miR-636 can be used as a biomarker to estimate the risk for the patient with chronic kidney disease suffering from urothelial carcinoma.

1.3 Logistic Regression Analysis

As above section 1.2, the logarithmic values of the formula 2^{-ΔCT} in the CKD group and the UC group were converted into the expression levels, and the expression levels were performed the model prediction by Logistic regression analysis. The predictive probability of urothelial carcinoma with the best sensitivity and specificity was selected as a cutoff value, and the receiver operating characteristic curve was plotted. The area under curve (AUC) of ROC curve was calculated, and there's no discrimination when the AUC=0.5. The more the AUC value, the stronger the discrimination. Table 3 below showed that the area under curve of the ratio of these miRNAs expression levels were at least more than 0.58.

TABLE 3
Analysis of receiver operating characteristics

| Sample sources | miRNA | AUC | Cutoff |
|---|---|---|---|
| Urine | miR-1274 | 0.6127 | 34.61 |
| | miR-19a | 0.5989 | 0.0002243 |
| | miR-30 | 0.6119 | 3.798 |
| | miR-708 | 0.6556 | 2.235*10⁻⁷ |
| Blood | miR-155 | 0.6417 | 1.227 |
| | miR-19b | 0.6601 | 0.3685 |
| | miR-210 | 0.6405 | 1.797 |
| | miR-378 | 0.5829 | 0.7642 |
| | miR-636 | 0.6095 | 0.5524 |

Table 3 shows that if miR-1274 in the specimen is less than or equal to its corresponding cutoff value of 34.61, miR-19a in the specimen is more than or equal to its corresponding cutoff value of 0.0002243, miR-30 in the specimen is less than or equal to its corresponding cutoff value of 3.798, miR-708 in the specimen is more than or equal to its corresponding cutoff value of 2.235*10⁻⁷, miR-155 in the specimen is less than or equal to its corresponding cutoff value of 1.227, miR-19b in the specimen is less than or equal to its corresponding cutoff value of 0.3685, miR-210 in the specimen is less than or equal to its corresponding cutoff value of 1.797, miR-378 in the specimen is less than or equal to its corresponding cutoff value of 0.7642, miR-636 in the specimen is less than or equal to its corresponding cutoff value of 0.5524, the subject will be estimated having the risk of suffering from the urothelial carcinoma. Thus, the expression level of at least one of the miRNAs more or less than its corresponding cutoff value can be used to estimate the risk for the subject suffering from urothelial carcinoma.

### Comparative Example 1

The UC and CKD groups are the same as example 1 in the section 1.1, the detection method of miRNA expression levels in the CKD and UC groups are the same as example 1 in the section 1.2. Table 4 showed expression levels of nine miRNAs different from Table 3, and the miRNAs used as a biomarker for estimating a risk for a subject suffering from urothelial carcinoma was estimated. The analysis was performed in the same method as Example 1 in section 1.3, and the area under the receiver operating characteristic curve was calculated.

TABLE 4
Analysis of receiver operating characteristics

| Sample sources | miRNA | AUC |
|---|---|---|
| Urine | miR-126-3p | 0.52 |
| | miR-202-3p | 0.51 |
| | miR-210-3p | 0.5 |
| | miR-33b-5p | 0.52 |

As shown in Table 4, the values of AUC in above four miRNAs miR-126-3p, miR-202-3p, miR-210-3p, and miR-33b-5p were nearly 0.5 which were considered no discrimination. Therefore, not all of the miRNAs can be used as biomarkers to estimate the subject having high risk of suffering from urothelial carcinoma.

### EXAMPLE 2

Simultaneous Use of Four miRNAs Expression Level in the Prediction of Urothelial Carcinoma

The expression levels of four miRNAs (miR-1274, miR-19a, miR-30 and miR-708) in the CKD and UC groups from Example 1 were used, and the expression levels were performed for the model equation prediction by Logistic regression analysis.

Prediction score S=−2.061+(1.698*A)+(1.300*B)+(0.861*C)+(1.330*D) equation (1).

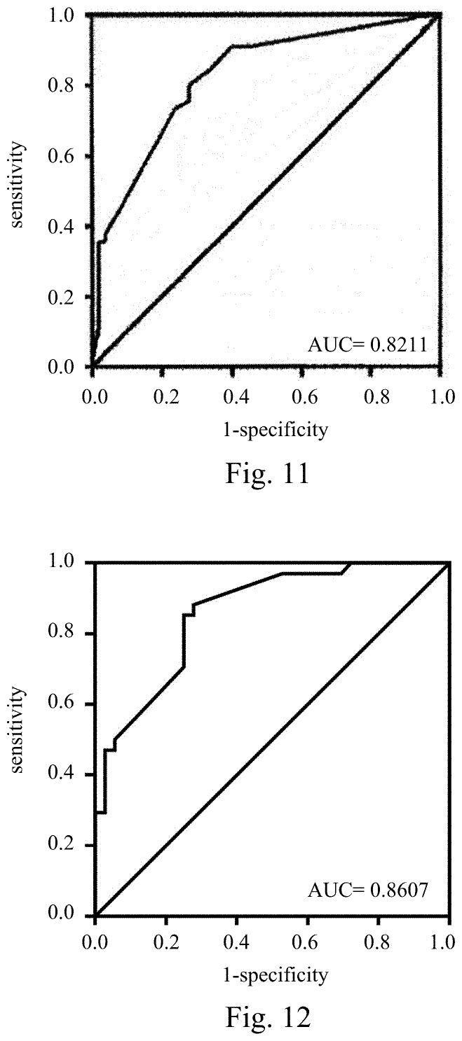

When the expression level of miR-1274 was less than or equal to 34.61, A=1, when the expression level of miR-1274 was more than 34.61, A=0; when the expression level of miR-19a was more than or equal to 0.0002243, B=1, when the expression level of miR-19a was less than 0.0002243, B=0; when the expression level of miR-30 was less than or equal to 3.798, C=1, when the expression level of miR-30 was more than 3.798, C=0; and when the expression level of miR-708 was more than or equal to 2.235*10⁻⁷, D=1, when the expression level of miR-708 was less than 2.235*10⁻⁷, D=0. The expression levels were analyzed by ROC curve, and the results were shown in Table 5 and FIG. 11.

TABLE 5
The relation of cutoff value, sensitivity
and specificity in ROC curve

| Cutoff value | Sensitivity | Specificity |
|---|---|---|
| >−0.7625 | 0.846 | 0.444 |
| >−0.2990 | 0.821 | 0.482 |
| >−0.0420 | 0.795 | 0.556 |
| >0.2515 | 0.795 | 0.667 |
| >0.4830 | 0.795 | 0.704 |
| >0.5700 | 0.718 | 0.778 |
| >0.8020 | 0.667 | 0.778 |
| >1.034 | 0.641 | 0.778 |
| >1.265 | 0.282 | 1.000 |
| >1.522 | 0.256 | 1.000 |
| >1.816 | 0.231 | 1.000 |
| >2.109 | 0.154 | 1.000 |
| >2.366 | 0.103 | 1.000 |
| >2.830 | 0.077 | 1.000 |

As shown in FIG. 11, the area under curve was 0.8211 indicating a high discriminating ability. As shown in Table 5 above, when the cutoff value is 0.4830, the sensitivity is 79.5% and the specificity is 70.4%. Therefore, simultaneous use of four miRNAs to predict urothelial carcinoma can obtain accurate prediction results.

### EXAMPLE 3

Simultaneous Use of Nine miRNAs Expression Level in the Prediction of Urothelial Carcinoma

The expression levels of nine miRNAs (miR-1274, miR-19a, miR-30, miR-708, miR-155, miR-19b, miR-210, miR-378, and miR-636) in the CKD and UC groups from Example 1 were used for the model equation prediction by Logistic regression analysis.

Prediction score S=−3.471+(1.258*A)+(0.590*B)+(0.327*C)+(1.042*D)−(0.561*E)+(1.605*F)−(0.172*G)+(0.413*H)+(1.541*I) equation (2).

When the expression level of miR-1274 was less than or equal to 34.61, A=1, when the expression level of miR-1274 was more than 34.61, A=0; when the expression level of miR-19a was more than or equal to 0.0002243, B=1, when the expression level of miR-19a was less than 0.0002243, B=0; when the expression level of miR-30 was less than or equal to 3.798, C=1, when the expression level of miR-30 was more than 3.798, C=0; when the expression level of miR-708 was more than or equal to 2.235*10⁻⁷, D=1, when the expression level of miR-708 was less than 2.235*10⁻⁷, D=0; when the expression level of miR-155 was less than or equal to 1.227, E=1, when the expression level of miR-155 was more than 1.227, E=0; when the expression level of miR-19b was less than or equal to 0.3685, F1, when the expression level of miR-19b was more than 0.3685, F=0; when the expression level of miR-210 was less than or equal to 1.797,G=1, when the expression level of miR-210 was more than 1.797, G=0; when the expression level of miR-378 was less than or equal to 0.7642, H=1, when the expression level of miR-378 was more than 0.7642, H=0; and when the expression level of miR-636 was less than or equal to 0.5524, I=1, when the expression level of miR-636 was more than 0.5524, I=0. The expression levels were analyzed by ROC curve, and the results were shown in Table 6 and FIG. 12.

TABLE 6
The relation of cutoff value, sensitivity
and specificity in ROC curve

| Cutoff value | Sensitivity | Specificity |
|---|---|---|
| >−0.9440 | 0.88 | 0.72 |
| >−0.5800 | 0.85 | 0.72 |
| >−0.3095 | 0.85 | 0.75 |
| >−0.1853 | 0.82 | 0.75 |
| >−0.0428 | 0.79 | 0.75 |
| >0.1440 | 0.76 | 0.75 |
| >0.4192 | 0.74 | 0.75 |

As shown in FIG. 12, the area under curve was 0.8607 indicating a high discriminating ability. As shown in Table 6 above, when the cutoff value is −0.3095, the sensitivity is 85% and the specificity is 72%. Therefore, simultaneous use of nine miRNAs to predict urothelial carcinoma can obtain more accurate prediction results comparing to simultaneous use of four miRNAs.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method for estimating a risk for a subject suffering from an urothelial carcinoma, the method comprising:
measuring an expression level of at least one micro ribosomal nucleic acid (miRNA) in a sample of the subject, wherein the miRNA is selected from the group consisting of miR-1274, miR-19a, miR-30, and miR-708; and
comparing the expression level of the miRNA in the sample to that of a same miRNA of a control, wherein a decrease in the expression level of the miRNA selected from the group consisting of miR-1274, miR-30 and miR-708 and/or an increase in the expression level of miR-19a in the sample from the subject, relative to that of the control, estimates the subject being of a risk of suffering from the urothelial carcinoma.

2. The method of claim 1, wherein the control is obtained from a group of subjects that does not have the urothelial carcinoma.

3. The method of claim 1, wherein the subject is a patient receiving peritoneal dialysis.

4. The method of claim 1, wherein the sample comprises ascites, blood, urine, feces, gastric juice, bile, or a combination thereof.

5. A method for estimating a risk of an urothelial carcinoma for a subject with chronic kidney disease, the method comprising:
measuring an expression level of at least one miRNA in a sample of the subject with chronic kidney disease, wherein the miRNA is selected from the group consisting of miR-1274, miR-19a, miR-30, miR-708, miR-155, miR-19b, miR-210, miR-378, and miR-636; and
comparing the expression level of the miRNA in the sample to that of a same miRNA of a control, wherein a decrease in the expression level of the miRNA selected from the group consisting of miR-1274, miR-30, miR-155, miR-19b, miR-210, miR-378, and miR-636 and/or an increase in the expression level of the miRNA selected from the group consisting of miR-19a and miR-708 in the sample from the subject with chronic kidney disease, relative to that of the control, estimates the subject being of a risk of suffering from the urothelial carcinoma.

6. The method of claim 5, wherein the control is obtained from a group of subjects with chronic kidney disease that does not have the urothelial carcinoma.

7. The method of claim 5, wherein the subject with chronic kidney disease is a patient receiving peritoneal dialysis.

8. The method of claim 5, wherein the sample comprises ascites, blood, urine, feces, gastric juice, bile, or a combination thereof.

9. A method for estimating a risk for a subject suffering from an urothelial carcinoma, the method comprising:
measuring expression levels of a plurality of miRNAs in a sample of the subject, wherein the plurality of miRNAs comprise miR-1274, miR-19a, miR-30, and miR-708;
calculating the expression levels of the plurality of miRNAs to obtain a prediction score S;
providing a reference value S0; and
estimating the risk for the subject suffering from the urothelial carcinoma, wherein when the prediction score S is more than or equal to the reference value S0, the subject is estimated having the risk of suffering from the urothelial carcinoma.

10. The method of claim 9, wherein the prediction score S is obtained by calculating the expression levels of the plurality of miRNAs with an equation below:
prediction score S=−2.061+(1.698*A)+(1.300*B)+(0.861*C)+(1.330*D) equation (1),
wherein when the expression level of miR-1274 is less than or equal to 34.61, A=1, when the expression level of miR-1274 is more than 34.61, A=0; when the expression level of miR-19a is more than or equal to 0.0002243, B=1, when the expression level of miR-19a is less than 0.0002243, B=0; when the expression level of miR-30 is less than or equal to 3.798, C=1, when the expression level of miR-30 is more than 3.798, C=0; and when the expression level of miR-708 is more than or equal to 2.235*10⁻⁷, D=1, when the expression level of miR-708 is less than 2.235*10⁻⁷, D=0.

11. The method of claim 9, wherein the step of providing the reference value S0 comprises:
measuring expression levels of a plurality of miRNAs in samples of a group of subjects with and without the urothelial carcinoma, wherein the plurality of miRNAs comprise miR-1274, miR-19a, miR-30, and miR-708; and
calculating the expression levels of the plurality of miRNAs in the samples of the group of subjects with and without the urothelial carcinoma by a receiver operating characteristic curve to obtain a cutoff value as the reference value S0.

12. The method of claim 9, wherein the plurality of miRNAs further comprise miR-155, miR-19b, miR-210, miR-378, and miR-636.

13. The method of claim 12, wherein the prediction score S is obtained by calculating the expression levels of the plurality of miRNAs with an equation below:
prediction score S=−3.471+(1.258*A)+(0.590*B)+(0.327*C)+(1.042*D)−(0.561*E)+(1.605*F)−(0.172*G)+(0.413*H)+(1.541*I) equation (2),
wherein when the expression level of miR-1274 is less than or equal to 34.61, A=1, when the expression level of miR-1274 is more than 34.61, A=0; when the expression level of miR-19a is more than or equal to 0.0002243, B=1, when the expression level of miR-19a is less than 0.0002243, B=0; when the expression level of miR-30 is less than or equal to 3.798, C=1, when the expression level of miR-30 is more than 3.798, C=0; when the expression level of miR-708 is more than or equal to 2.235*10⁻⁷, D=1, when the expression level of miR-708 is less than 2.235*10⁻⁷, D=0O; when the expression level of miR-155 is less than or equal to 1.227, E=1, when the expression level of miR-155 is more than 1.227, E=0; when the expression level of miR-19b is less than or equal to 0.3685, F1, when the expression level of miR-19b is more than 0.3685, F=0; when the expression level of miR-210 is less than or equal to 1.797, G=1, when the expression level of miR-210 is more than 1.797, G=0; when the expression level of miR-378 is less than or equal to 0.7642, H=1, when the expression level of miR-378 is more than 0.7642, H=0; and when the expression level of miR-636 is less than or equal to 0.5524, I=1, when the expression level of miR-636 is more than 0.5524, I=0.

14. The method of claim 12, wherein the step of providing the reference value S0 comprises:
measuring expression levels of a plurality of miRNAs in samples of a group of subjects with and without the urothelial carcinoma, wherein the plurality of miRNAs comprise miR-1274, miR-19a, miR-30, miR-708, miR-155, miR-19b, miR-210, miR-378, and miR-636; and
calculating the expression levels of the plurality of miRNAs in the samples of the group of subjects with and without the urothelial carcinoma by a receiver operating characteristic curve to obtain a cutoff value as the reference value S0.

15. The method of claim 9, wherein the subject is a patient receiving peritoneal dialysis or with chronic kidney disease.

16. The method of claim 9, wherein the sample comprises ascites, blood, urine, feces, gastric juice, bile, or a combination thereof.

17. A kit for estimating a risk for a subject suffering from an urothelial carcinoma, the kit comprising:
at least one reagent, the reagent using for detecting an expression level of at least one miRNA in a sample of the subject, wherein the miRNA is selected from the group consisting of miR-1274, miR-19a, miR-30, miR-708, miR-155, miR-19b, miR-210, miR-378, and miR-636.

18. The kit of claim 17, wherein the kit comprises a plurality of reagents using for detecting expression levels of the miRNAs in the sample of the subject, wherein the miRNAs are miR-1274, miR-19a, miR-30, and miR-708.

19. The kit of claim 17, wherein the kit comprises a plurality of reagents using for detecting expression levels of the miRNAs in the sample of the subject, wherein the miRNAs are miR-1274, miR-19a, miR-30, miR-708, miR-155, miR-19b, miR-210, miR-378, and miR-636.

20. The kit of claim 17, wherein the reagent comprises a pair of primers, a probe, or a combination thereof.
